(19) United States
(12) Patent Application Publication
(10) Pub. No.: US 2025/0144122 A1
(43) Pub. Date: May 8, 2025

(54) Scent-Changing Ointment & Method of Making The Same

(71) Applicants: Roberto Colon-Cruz, Luquillo, PR (US); Astrid Velez-Henao, Luquillo, PR (US)

(72) Inventors: Roberto Colon-Cruz, Luquillo, PR (US); Astrid Velez-Henao, Luquillo, PR (US)

(21) Appl. No.: 18/502,157

(22) Filed: Nov. 6, 2023

(51) Int. Cl. A61K 31/00; A61K 9/00; A61K 9/06; A61K 31/05; A61K 31/125; A61K 31/618; A61K 35/644; A61K 36/898; A61K 47/44

(52) U.S. Cl. CPC A61K 31/658; A61K 9/0014; A61K 9/06; A61K 31/05; A61K 31/125; A61K 31/618; A61K 35/644; A61K 36/898; A61K 47/44

(57) ABSTRACT

A topical ointment that is useful for treating dry skin and that is also capable of gradually changing its scent or aroma as time goes by.

### STATEMENT REGARDING FEDERALLY SPONSORED RESEARCH AND DEVELOPMENT

N/A

### RELATED APPLICATIONS

N/A

### BACKGROUND OF THE DISCLOSURE

### Field of the Invention

The present application relates to a topical ointment that is useful for treating dry skin and is also capable of gradually changing its scent or aroma as time goes by.

### BACKGROUND

The influence of fragrances such as perfumes and room fresheners on the psychophysiological activities of humans has been known for a long time, and its significance is gradually increasing in the medicinal and cosmetic industries. The sense of smell plays an important role in the physiological effects of mood, stress, and working capacity. For example, studies have shown that vanilla has therapeutic effects on stress and anxiety, making this scent especially beneficial for people who struggle with these issues. Singletary, Keith W. Vanilla, Potential Health Benefits. Nutrition Today. 55 (4): p 186-196, 7/8 2020. Based on the previous studies, it can be concluded that fragrances directly and/or indirectly affect the psychological and physiological conditions of humans.

It should be noted that the term “olfactory fatigue” refers to the temporary inability to distinguish a particular odor after a prolonged exposure to an airborne compound. Olfactory fatigue occurs when a person's sense of smell becomes desensitized to a particular fragrance after prolonged exposure. This is a common phenomenon that can happen to anyone, and it can be reversed by taking a break from the fragrance for a period of time. As such, olfactory fatigue represents an obstacle for industries that incorporate a particular scent into their products. The subject disclosure addresses this obstacle by describing a topical ointment that is capable of gradually changing its scent or aroma as time goes by, thereby reducing the effects of olfactory fatigue.

As evidenced by the great number of perfumes, soaps, and cosmetic creams available in the market, as well as the mass advertising they receive, the scent of a skin product has a great influence on their desirability. This is also true with respect to topical analgesic ointments or creams since many people dislike the strong odor that these topical creams impart. Accordingly, it would be advantageous to have a topical cream or ointment having the capability of progressively changing its scent into a desirable odor or aroma as time goes by. The subject disclosure, therefore, addresses this need by disclosing an enhanced topical ointment that changes its scent as time goes by, thus making the ointment more appealing to potential users when applying it to the skin.

While certain aspects of conventional technologies have been discussed to facilitate disclosure of the invention, Applicant in no way disclaims these technical aspects, and it is contemplated that the claimed invention may encompass one or more of the conventional technical aspects discussed herein. In this specification where a document, act, or item of knowledge is referred to or discussed, this reference or discussion is not an admission that the document, act, or item of knowledge or any combination thereof was at the priority date, publicly available, known to the public, part of common general knowledge, or otherwise constitutes prior art under the applicable statutory provision; or is known to be relevant to an attempt to solve any problem with which this specification is concerned.

### SUMMARY

The present disclosure relates to a Hemp/CBD oil-based ointment that is useful for treating dry skin and has the capability of gradually changing its scent or aroma. The claimed topical ointment is a composition that comprises 30% of percentage by weight of Methyl Salicylate, 3% of percentage by weight of Menthol, 6% of percentage by weight of Camphor and 25 mg of CBD Hemp Microspheres per 100 grams of ointment. Additionally, the topical ointment comprises vanilla oil, organic beeswax, thymol, and organic coconut oil. The coconut oil allows the ointment to be transferred or applied, while the bees wax holds the composition (low-melt wax), sealing the surface and allowing for the skin to build up its natural moisture level by minimizing moisture loss on the surface, plus adding a fragrance agent, provides a method of sooth of moisturizing, soothing, or healing of external tissues comprising applying an effective amount of the composition to the tissue. Furthermore, the invention provides a method of moisturizing or alleviating dry, chapped, or irritated skin comprising applying an effective amount of the composition. The proposed ratios were selected due to their physical and chemical characteristics to allow the disclosed ointment to melt on your skin and not in its jar; and because of the observed arrays of odors from menthol/camphor to vanilla scent, to coconut oil. Thymol was added as an antiseptic with the dual purpose of protecting the formulation and to obtain the antiseptic characteristics in the skin such in the case of mosquito bites. The subject disclosure provides a novel transformation of pleasant odorant, scent, aroma, odor compositions and a novel method of imparting a pleasant odorant, scent, aroma, odor to a composition. These and other objects and advantages of the invention will become obvious from the following detailed description.

The present disclosure may address one or more of the problems and deficiencies of the prior art discussed above. However, it is contemplated that the disclosure may prove useful in addressing other problems and deficiencies in several technical areas. Therefore, the claimed invention should not necessarily be construed as limited to addressing any problems or deficiencies discussed herein.

### BRIEF DESCRIPTION OF THE DRAWINGS

These and other features, aspects, and advantages of the present invention will become better understood regarding to the following description, appended claims and accompanying drawings where:

FIG. 1 shows a table describing the ingredients that form part of the claimed topical ointment along with their corresponding melting point range, boiling point, and vapor pressure.

FIG. 2 shows a method of making the claimed topical ointment.

FIG. 3 shows the equipment used to make the claimed topical ointment.

### DETAILED DESCRIPTION

In the Summary above and the Description below, the term “comprises” and grammatical equivalents thereof are used herein to mean that other components, structures, steps, etc. are optionally present. For example, a composition “comprising” (or “which comprises”) elements A, B, and C can consist of (i.e., contain only) elements A, B, and C, or can contain not only elements A, B, and C, but also one or more other elements.

The term “at least” followed by a number is used herein to denote the start of a range beginning with that number (which may be a range having an upper limit or no upper limit, depending on the variable being defined). For example, “at least 1” means 1 and/or more than 1.

While the specification will conclude defining the features of exemplary embodiments of the disclosure that are regarded as novel, it is believed that the disclosure will be better understood from a consideration of the following description in conjunction with the figures, in which like reference numerals are carried forward.

The present disclosure relates to a Hemp/CBD oil-based ointment that is useful for moisturizing or treating dry skin; and has the capability of gradually changing its scent or aroma. In particular, upon contact with the skin, the ointment initially has a scent of camphor and/or menthol. After a few minutes the scent changes to vanilla; and lastly, the smell gradually changes into a coconut oil scent. The CBD compound may be present as a substantially pure isomer, isomers, acids, salts, and salts of isomer or a racemic mixture of isomers. It should be noted, however, that while the cannabidiol selected in the preferred embodiment of the disclosure is CBD; in other embodiments, the cannabidiol may be selected from the group consisting of CBC, CBG, CBN, CBL, CBDV, and combinations thereof.

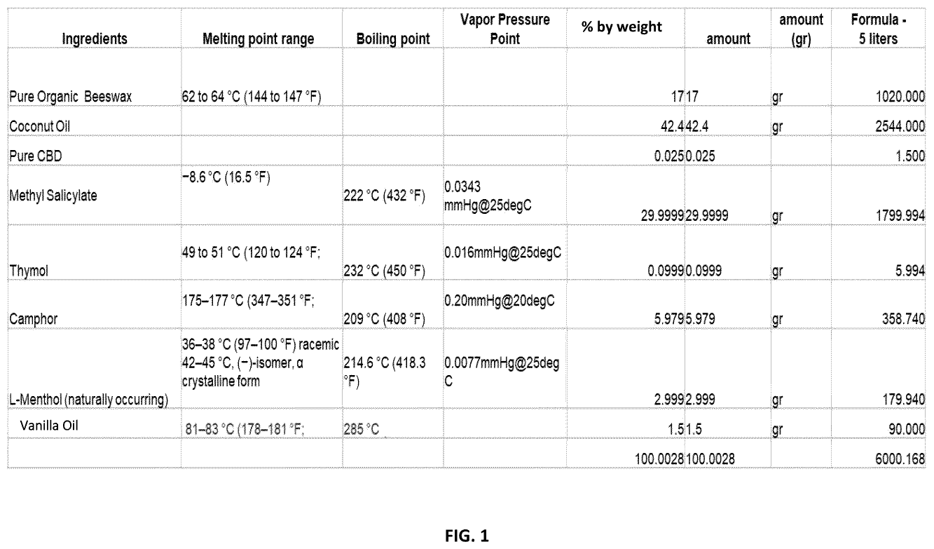

The table shown in FIG. 1 describes the ingredients that form part of the claimed topical ointment along with their corresponding melting point range, boiling point, vapor pressure, % by weight of each component of the formulation.

The subject disclosure may comprise disinfectant formulations or one or more agents that impart a pleasant odor or scent thereto (fragrance agent). Non-limiting examples of agents imparting a pleasant odor and/or flavor, and/or enhancing properties comprise essential oils, carvacrol, good Samaritan oil, cymene, cineol, cinnamon, eugenol, thymol, menthol, citral, vanilla and limonene. Further suitable examples of such agents are within the capacity of a skilled technician.

As noted above, the claimed topical ointment will have an initial smell of camphor and menthol. Within minutes, the smell of the claimed topical ointment will then slowly transform into a fresh, light and refreshing smell of vanilla. Lastly, the topical ointment imparts a coconut oil aroma for a few more hours. The gradual change in scent or aroma of the topical ointment arises from the difference in vapor pressure from each odor containing ingredient. In particular, due to its high vapor pressure, camphor has a natural tendency to sublimate or change from a solid to a gaseous state at room temperature, albeit rather slowly. When camphor is mixed with menthol, however, the resulting mixture is forced to go first through a liquid state and then into the gaseous state. Due to the lower melting point of menthol visa vis camphor, the former evaporates faster than the latter. Subsequently, after both camphor and menthol have evaporated, the ointment imparts a vanilla scent since vanilla oil has a higher boiling point vis a vis camphor and menthol. Lastly, after the vanilla oil gradually evaporates, the ointment imparts the aroma of coconut oil, which is the remaining odor containing ingredient of the ointment.

It should be noted that chemical changes elicit a transformation in the chemical make-up of different substances to form a new substance. For camphor, however, its natural tendency is to sublimate, or go directly from a solid to a gas at room temperature, albeit rather slowly. When it is mixed with menthol, the new substance adulterates the camphor, and the mixture forces it to go through the liquid state. In other words, adding a foreign substance will cause the substance to become impure and change its physical properties, or changes that do not change the composition of the substance providing a eutectic system.

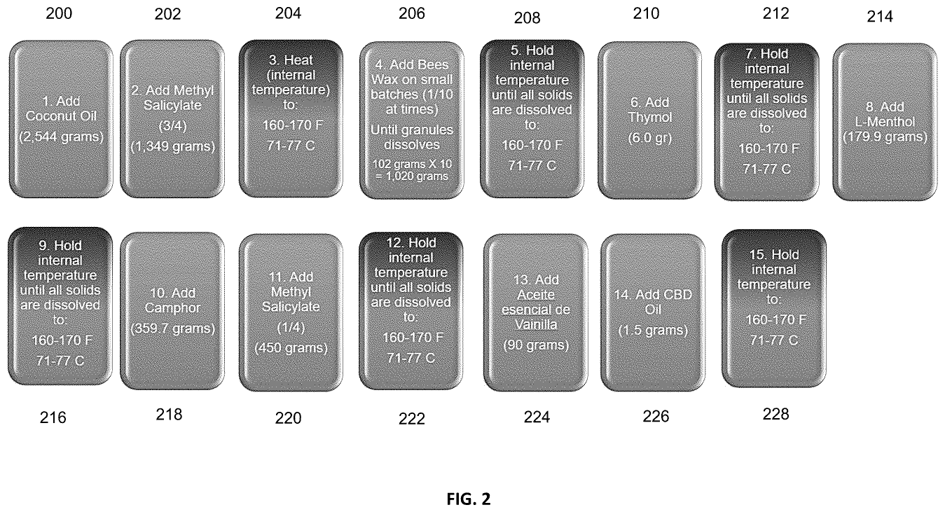

Moreover, as shown in FIG. 2, the method of making the topical ointment described herein comprises the step 200 of adding to a reactor 2,544 g of coconut oil; followed by adding 1,349 g of methyl salicylate, as shown in step 202; and heating the mixture of these two substances up to the range of 71-77° C. until fully dissolved, as shown in step 204. Next, 1,020 g of beeswax are added in small batches ( 1/10 at times) to the mixture until granules dissolve, as shown in step 206. The internal temperature is held until all solids are dissolved to 71-77° C., as shown in step 208. Next, 6 g of thymol are added, and the internal temperature is held until all solids are dissolved to 71-77° C., as shown in steps 210 and 212. Next, 179.9 g of L-Menthol are added, and the temperature is held until all solids are dissolved to 71-77° C., as shown in steps 214 and 216. Next, 359.7 g of Camphor are added, as shown in step 218, followed by 450 g of methyl salicylate, and the temperature is held until all solids are dissolved to 71-77° C., as shown in steps 220 and 222. Then, 90 g of vanilla oil is added, as shown in step 224, followed by 1.5 g of CBD Oil (microencapsulated), and the temperature is held until all solids are dissolved to 71-77° C., as shown in steps 226 and 228. It should be noted that CBD and vanilla oil must be added at the end to avoid long-term exposure to high temperatures. Vanilla is the last scent due to its vapor pressure within the general composition. The materials are first in solution; they are all in liquid form and flowing while the formulation is heated. When the vanilla oil is added, the formulation is still liquid. When the matrix changes from a liquid to a semi-solid when it cools down, the process is completed. In a semisolid state, the formulation has a degree of diffusion.

The proposed ratios were selected due to their physical and chemical characteristics which allow the disclosed topical ointment to melt on an individual's skin and not while stored in a container; and because of the observed arrays of odors from menthol/camphor to vanilla scent, and finally to coconut oil. Thymol was selected due to its antiseptic properties, which serve the dual purpose of protecting the formulation; and protecting the skin from minor injuries, such as mosquito bites.

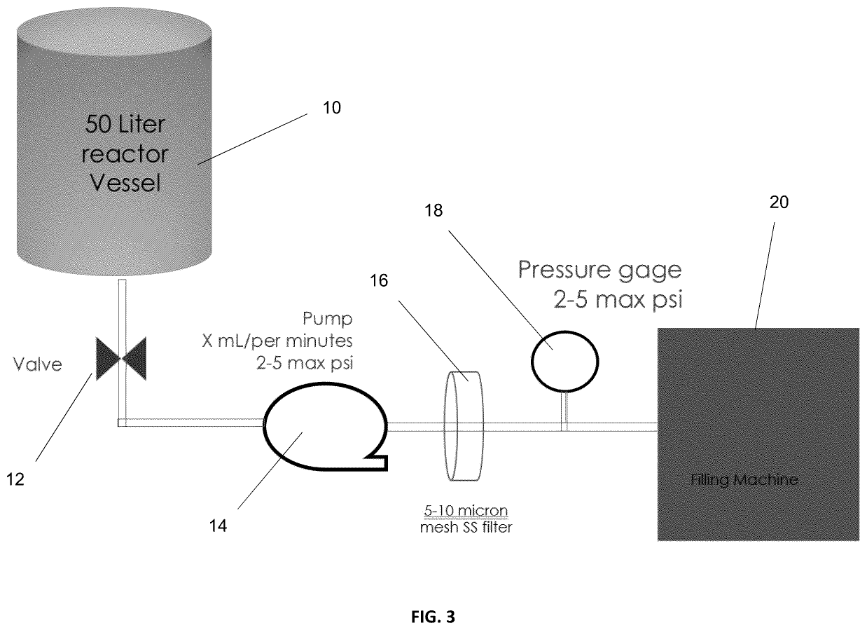

FIG. 3 shows the process of packaging the claimed topical ointment. As shown in FIG. 3, the ingredients are first added to a reactor 10, where the ingredients are heated as discussed above. The mixture is then led, via the valve 12, to a pump 14, which causes the mixture to pass through a 5-10 micron SS filter 16 (to capture any impurities), which leads directly to a filling machine 20, where the resulting ointment is dispensed into the final packaging. The pressure throughout the production process should be maintained in the range of 2-5 max psi, as shown by the pressure gauge 18. It should be noted that the active ingredients of the claimed topical ointment are methyl salicylate and CBD; that the aromatic components of the claimed topical ointment claimed topical ointment are Menthol, Camphor, and Vanilla; the diluents or excipients are coconut oil and beeswax; and that the antiseptic component is Thymol.

The particular order of the process is selected for the well-being of the process and the ease of adding the various components. the process starts with coconut oil and methyl salicylate (the solvent) to provide a warm bath and create a matrix; then the solid components are added, and a part of the liquid components (the solvent) is used to clean the containers and the walls of the reactors; and finally, CBD and vanilla oil are added to avoid long-term exposure to high temperatures. It is a practical and convenient way of doing the process on a small scale that can be transferred to a large scale. It is important that the process protects the formulation and the compounds that are more volatile, such as vanilla and CBD, once the volatile compounds enter the matrix. This is accomplished because the physical properties of the other less volatile compounds in the matrix cause the more volatile compounds to not filter or evaporate so easily. For example, coconut oil and methyl salicylate are non-volatile and as such they make up a good matrix to protect the properties of the more volatile components.

In sum, the chemical integrity of the materials that make up the formulation is preserved. Temperatures higher than 77° C. are not used; this protects all the materials in the formulation. The vanilla oil is added last because it is the material most likely to evaporate first. On the other hand, vanilla oil has a very high vapor pressure. This is why the last scent to be released from the ointment when applied to the skin. As noted, the organic matrix is in a semi-solid state and the materials that make up the formulation are embedded within the matrix. The vanilla oil is trapped in the matrix along with the other molecules from the other materials. For the evaporation process to occur, what is on the surface and what is inside the matrix has to be taken into consideration, what is on the surface has first-order evaporation, and for what is inside the matrix, evaporation is of zero order, and there has to be diffusion. When the ointment is applied to the skin, as soon as the material receives heat from the body, everything in the matrix behaves as if it were on the surface because of the change in temperature upon contact with the skin. Upon contact with the skin, the first scents to emanate are menthol and camphor, because they are of the first order. The same thing happens when the formulation is inside of a container, what is on the surface tends to evaporate faster because it is of the first order and what is inside the matrix stays longer inside and evaporates much later.

Permeability and solubility also explains why the different scents are being released progressively. Coconut oil and methyl salicylate are quite permeable, this allows the other materials to penetrate the skin as well. The penetration process is called permeability. The formulation is made with compounds that promote the permeability of the other compounds through the skin.

The ointment is very stable; it melts but it will not separate. The person uses the product as soon as the matrix touches the skin. It bonds with the skin, it moisturizes, the molecules pass through the skin, at the same time it does not leave a sticky residue because while it is in small amounts that are absorbed through the skin, you do not feel the sticky sensation. At the same time, it starts first smelling the menthol and camphor, then that smell is completely gone because they have high vapor pressure. Then the smell of vanilla is gone; and then what remains is the faint smell of coconut.

The ointment is all made in a reactor 10 with heat applied to it. The reactor 10 circulates water on the walls and with a motor above (impeller) for circulation. The reactor has a lamp, it has the materials in motion, it has an impeller that makes revolutions per minute, the materials are added from above, they are mixed and once mixed, a peristaltic pump is used, and the containers are filled with the ointment.

The inventor has collected anecdotal, testimonial, and uncontrolled medical study evidence showing that the claimed topical ointment worked best in the proportions shown in FIG. 1. Accordingly, the claimed topical ointment comprises 29.9%±10% of percentage by weight of Methyl Salicylate, 2.9%±5% of percentage by weight of Menthol, 5.9%±5% of percentage by weight of Camphor and 0.025%±2% percentage by weight or 25 mg of CBD Hemp Microspheres. Additionally, the topical ointment comprises 1.5% 3% of percentage by weight of vanilla oil, 17%±10% of percentage by weight of organic beeswax, 0.09%±2% of percentage by weight of thymol and 42%±10% of percentage by weight of organic coconut oil.

It should be noted that the disclosed topical ointment may also be prepared with lavender oil, shea butter, almond oil, avocado oil, neem oil, seed oil, sunflower oil, or other natural oils instead of coconut oil, but in the same percentage by weight corresponding to coconut oil described above. Additionally, instead of vanilla oil, the topical ointment may also be prepared with linalool, limonene, eucalyptol, or other essential oils (in the same percentage by weight corresponding to vanilla oil described above). Similarly, carvacrol, citral, eucalyptus may be used instead of thymol (in the same percentage by weight corresponding to thymol described above).

Moreover, beeswax can be substituted with animal, vegetable, and/or petroleum waxes such as, paraffin or soy wax (in the same percentage by weight corresponding to beeswax described above). Lastly, instead of camphor or menthol, the disclosed topical ointment may also be prepared with salicylate, diclofenac, ketoprofen, salicylamide, nicotinate esters or phenol (in the same percentage by weight corresponding to camphor and menthol described above).

What follows is a description of the benefits of each of the ingredients of the disclosed topical ointment:

Hemp/CBD spheres are liposomes that have several membranes/layers within each other. Each membrane bursts at different times, one after the other allowing the skin to benefit from small doses of the active ingredient for all-day delivery into the skin. Encapsulation of CBD equals improved effectiveness, easier solubilization and the encapsulated ingredient is more stabilized in the bottle, and only bursts after absorption into the skin. Encapsulated CBD skin delivery technology allows active ingredient to become more efficacious and less irritating. These microscopic spheres release one layer at a time so that you get little bursts of CBD. Each layer releases the encapsulated ingredient in time-released intervals for delivery into the skin, increasing permeation with liposomal encapsulation, eliminates or reduce irritation and optimizes dosage with greater concentration and residence time in the epidermis and dermis. This results in improved penetration, protection of the outer skin layer and reduced toxicity if the ingredient is a medicine.

The skin has its own endocannabinoid system (CB1 and CB2) that regulates hormone and protein production, including cytokines, which are involved in the immune response, and cannabinoid lipids, which regulates homeostasis and skin inflation. CB1 receptor mediate the inhibition of neurotransmitter release (involved in the skin), while CB2 modulate cytokine release (usually involved with the anti-inflammatory effect). The skin possesses a robust capacity to synthesize and respond to cannabinoids, like CBD receptor and can recognize and bind with CBD which in turn can reduce pain by impacting these receptors activity, reducing inflammation and interacting with neurotransmitters. Thus, endocannabinoids and their receptors constitute part of an adaptive system to regulate cutaneous inflammation.

Methyl Salicylate is a natural product of many species of plants. Methyl Salicylate Properties. (n.d.). World of Chemicals. Retrieved Jul. 28, 2023. Some of the plants which produce it are called wintergreens, hence the common name. Methyl Salicylate. (n.d.). Ataman Chemicals. Methyl salicylate is an analgesic. Methyl salicylate is an active ingredient in many over-the-counter pain-relief ointments. It is one of a group of anti-inflammatory chemicals known collectively as salicylates because salicylic acid is their shared root compound. Aspirin—salicylic acid with an acetyl group attached (thus its formal chemical name, acetylsalicylic acid)—is the best known of the salicylates. Topical analgesics containing methyl salicylate suppress pain by blocking the enzymes involved in the production of prostaglandins, which signal inflammation and cause pain. The health benefits of wintergreen oil are in the analgesic, anti-rheumatic, anti-spasmodic and astringent properties it contains. The oil is said to relieve pain by way of the methyl salicylate compound, which causes the treated, or affected area of the body to experience a feeling of numbness, to help with blood circulation and to promote the warming of the affected area. The mechanism of action by which methyl salicylate expresses topical analgesia is not fully understood. Current literature indicates that methyl salicylate has both stimulatory and inhibitory actions on TRPV1 channels and suggests that the latter action may partly underlie the analgesic effects of methyl salicylate independent of inhibition of cyclooxygenases in vivo. Is used topically as a counterirritant. On topical application, it is absorbed through the skin and is applied for the relief of pain in rheumatic conditions and painful muscle or joints. One of the most popular over-the counter group of topical agents is the salicylate-containing anti-inflammatory formulation, recommended for use in wide range of painful or inflammatory conditions in muscles and joints. It has been shown that first-pass metabolism exists in the skin with esterase rapidly hydrolyzing salicylate esters to release the active salicylate in both the epidermis and dermis. The bottom line: Products that contain methyl salicylate might provide some pain relief.

Menthol is an organic compound made synthetically or obtained from peppermint, or other mint oils, that produces a feeling of cooling. The natural form of menthol ((L)-menthol) exists as one pure stereoisomer and is the preferred form for analgesic effects. Menthol's ability to chemically trigger the cold-sensitive TRPM8 receptors in the skin is responsible for the well-known cooling sensation that it provokes when inhaled, eaten, or applied to the skin. Menthol has analgesic properties that are mediated through a selective activation of K-opioid receptors. Typically, ice is applied to the skin to create a cold response in order to reduce pain because cold reduces the pain threshold. Menthol creates a chemical action on cold receptors rather than a physical action, resulting in a cold response. Patel and colleagues provide an excellent review of the mechanisms behind menthol. L Patel T, Ishiuji Y, Yosipovitch G. Menthol: a refreshing look at this ancient compound. J Am Acad Dermatol 2007; 57 (5): 873-878.); incorporated herein by reference. Similar to ice, topical application of menthol in a 3.5% gel reduces blood flow by 35% within 60 seconds of application and remains-20% reduced at 10 minutes after application. (Olive J L, Hollis B, Mattson E, Topp R. Vascular conductance is reduced after menthol or cold application. Clin J Sport Med. 2010; 20 (5): 372-376); incorporated herein by reference. Recently, Topp and colleagues noted decreased blood flow in both lower limbs after application to one limb, suggesting a possible systemic mechanism of topical menthol. Int J Sports Phys Ther. 2011 June; 6 (2): 83-91.

As discussed in US Patent No. U.S. Pat. No. 8,889,659 B2, incorporated herein by reference, studies have shown that (l)-menthol (natural menthol derived from peppermint oil) was able to increase pain threshold whereas (d)-menthol (synthetic menthol) was completely devoid of any analgesic effect. Menthol is an active ingredient in most of the traditional rub-in products that elicit a cooling sensation. Menthol, on topical application, dilates the blood vessels causing a sensation of coldness followed by an analgesic effect. Id. Menthol also acts as a penetration enhancer. It increases the penetration of drugs when applied on the skin to give a faster onset of action. Menthol functions as a penetration enhancer either due to its vasodilatation property or since menthol readily penetrates the skin, it may reversibly change barrier-structures in the stratum corneum without affecting blood circulation at the site of application.

As also discussed in US Patent No. U.S. Pat. No. 8,889,659 B2, camphor is a naturally occurring compound that is used as a major active ingredient of ointments and liniments supplied as topical analgesics. Camphor is readily absorbed through the skin, is highly volatile and readily absorbed through the skin. It produces a cool sensation and can under certain circumstances act as a mild local anesthetic. Id. Camphor is readily absorbed through the skin and produces a feeling of cooling like menthol and can act as a local anesthetic substance. Id. There are anti-itch and cooling gels with camphor as the active ingredient. Id. When applied externally, camphor can numb the nerve endings. Id. The nerve endings then no longer transmit the sensation of pain. Camphor has recently been shown to activate TRPV3, and it has been demonstrated that camphor also activates heterologous expressed TRPV1, requiring higher concentrations than capsaicin. Id. The camphor-induced desensitization of TRPV1 and block of TRPA1 may underlie the analgesic effects of camphor. Id. Camphor oil was traditionally massaged into sprains and sore muscles and joints for pain relief, and most modern herbalists agree that this is the best use for pure camphor oil. Id. Camphor has been shown to help ease inflammation. It is readily absorbed when applied topically, making it a particularly effective treatment for arthritic and rheumatic joint pain. Id. Camphor can exist in two enantiomers: (+)- or (R)-camphor (shown), the predominant natural isomer, and (−)- or (S)-camphor.

Vanilla Essential Oil can be attributed to its properties as an antioxidant, aphrodisiac, anticarcinogenic, febrifuge, antidepressant, sedative, tranquilizing and relaxing substance. Pure Natural Cosmetics. (n.d.). Pure Cosmetics. The Essential Oil of Vanilla is extracted by solvent extraction of a resinous substance obtained from fermented vanilla beans. Id. Its main components are Acetic Acid, Capric Acid, Eugenol, Furfural, Isobutyric Acid and Vanillin Hydroxybenzaldehyde. Apart from its widespread use as a flavoring agent in certain organic foods, beverages and pharmaceutical industries as well as in culinary endeavors, the essential oil of vanilla has found extensive use in the world of medicine as well.

Beeswax is commonly used in various skin care products to improve the condition and texture of the skin. Beeswax Health Benefits. (n.d.). Diet Health Club. It also has a number of benefits in treating skin problems like acne, eczema, and dry skin. Id. Beeswax helps in locking skin moisture, fostering the skin cells and protecting the skin from environmental damage. Id. It contains powerful anti-bacterial, anti-inflammatory, and antioxidant properties that are all beneficial to the skin. Id. Besides offering your skin a natural moisture and glow, the anti-allergenic and anti-inflammatory properties of beeswax can actually soothe easily irritated skin, making it one of the best skin care ingredients for those who suffer from allergies or other skin problems like rosacea or eczema. Along with being a good alternative skin care ingredient for those with allergies, the anti-germicidal properties of beeswax have been found to help heal minor skin cuts, abrasions, scrapes and wounds. Beeswax also acts as a layer of protection when applied to the skin, protecting the skin from environmental irritants and even the weather.

The effectiveness of beeswax for acne treatment and control is well known. Id. It has strong antiseptic, healing, and anti-inflammatory properties that are effective in the treatment of acne. Id. Beeswax contains vitamin A, which is an added advantage in the treatment of acne. It is also an excellent skin softener and emollient that helps maintain a smooth skin texture after acne elimination. Id. The ability of beeswax to lock in moisture has made it an important ingredient in a variety of lotions and creams for treating dry skin. Id. In addition, its rich vitamin A content and emollient properties help in the healthy development of skin cells. Id. It is a natural moisturizer that not only treats dry skin, but also helps prevent it. Id. Beeswax is a great treatment for skin problems like eczema that cause an itchy skin rash. Id. Eczema is a common skin problem especially during the colder months that results in extremely dry skin. Id. The medicinal properties of beeswax help in reducing eczema inflammation and prevent further infection. Id. The natural moisturizers in beeswax make it a good ointment for the lips. Id. Users can easily treat problems like cracked or chapped lips with the help of topical beeswax applications. Id.

Thymol, and its main natural source, thyme (Thymus vulgaris L.), are employed for their positive antioxidant, anti-inflammatory, local anesthetic, antinociceptive, cicatrizing, antiseptic, virucide, antibacterial, and antifungal properties as well as for their beneficial effects on the cardiovascular system. Marchese, A., Orhan, I.E., & Daglia, M. (2016). Antibacterial and antifungal activities of thymol: A brief review of the literature, Vol. 210, pp. 402-414. Thymol is widely used as a topical antiseptic agent in sore throat lozenges and sprays and as well as a topically applied skin exfoliant. Small amounts of Thymol are present in many consumer products that are commonly used, such as mouthwashes, sore throat lozenges, ear or nose drops, cold sore lotions, analgesic rubs and antiseptic lotions. Additionally, the source plant (thyme), from which thymol is extracted is acknowledged by FDA as generally recognized as safe (GRAS) (21 CFR 182.10, 21 CFR 182.20). Thymol is listed on four (4) of antimicrobial products for use against SARS-COV-2, under an emerging viral pathogens such as but not explicit to Norovirus and Rhinovirus. The phenolic compounds of natural origin as used in the present invention can be synthetically made by known methods within the capacity of a skilled technician, or can be obtained from plant oil extracts.

Coconut oil for skin care has been a staple in skin care products for quite some time. When at room temperature, it is solid and white, but melts into a clear liquid with very little heat. The use of coconut oil for skin care is common because it is extremely effective at hydrating the skin. Unlike most commercial moisturizers, coconut oil does not contain water or petroleum byproducts. Petroleum byproducts may become clogged in people's pores, causing acne and other unsightly blemishes. When using coconut oil for skin care, the probability of these negative factors can be reduced.

Coconut oil includes saturated fats. When applied on the skin, they keep it smooth to the touch. Coconut Oil-Composition (n.d.). Elly's Natural Skin Care. Retrieved Jul. 31, 2023, from https://ellysnaturalskincare.com/pages/benefits-of-coconut-oil. Due to the presence of these fats, coconut oil also retains the moisture content of the skin, as the fats eliminate moisture loss through the pores on skin. Id. Like coconuts themselves, coconut oil is rich in many proteins. Id. These proteins keep skin healthy and rejuvenated, both internally and externally. Id. Proteins also contribute to cellular health and tissue repair, along with a wide range of other essential activities within the body. Id. For any damaged cells on or near the skin, a healthy flow of proteins guarantees their replacement at a normal rate, whereas people with protein-deficient diets heal slower and often develop more obvious scars due to the extended healing time.

Lastly, it must be noted that the topical ointment compromises natural ingredients and contains no tetrahydrocannabinol (“THC”), petroleum, parabens, or phthalates. Additionally, it is gluten-free and GMO free.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in any future claim.

What is claimed is:

1. A scent-changing topical ointment, comprising:
29.9 wt. % of methyl salicylate;
2.9 wt. % of menthol;
5.9 wt. % of camphor;
0.025 wt. % or 25 mg of CBD hemp microspheres;
1.5 wt. % of vanilla oil;
17 wt. % of organic beeswax,
0.09 wt. % of thymol; and
42% wt. % of coconut oil.

2. The scent-changing topical ointment of claim 1, wherein the coconut oil is substituted with one of the following oils: lavender oil, shea butter, almond oil, avocado oil, neem oil, seed oil, or sunflower oil, each having the same percentage by weight as the coconut oil.

3. The scent-changing topical ointment of claim 1, wherein the vanilla oil is substituted with one of the following oils: linalool oil, limonene oil, eucalyptol oil, cinnamon oil, or Samaritan oil, each having the same percentage by weight as the vanilla oil.

4. The scent-changing topical ointment of claim 1, wherein the thymol is substituted with any of the following substances: carvacrol, citral, eucalyptus, each having the same percentage by weight as the thymol.

5. The scent-changing topical ointment of claim 1, wherein the beeswax is substituted with any of the following substances: paraffin or soy wax, each having the same percentage by weight as the beeswax.

6. The scent-changing topical ointment of claim 1, wherein the camphor is substituted with any of the following substances: salicylate, diclofenac, ketoprofen, salicylamide, nicotinate esters or phenol, each having the same percentage by weight as the camphor.

7. The scent-changing topical ointment of claim 1, wherein the menthol is substituted with any of the following substances: salicylate, diclofenac, ketoprofen, salicylamide, nicotinate esters or phenol, each having the same percentage by weight as the menthol.

8. A method of making a scent-changing topical ointment, comprising the steps of:
adding into a reactor 42% wt. % of coconut oil and 29.9 wt. % of methyl salicylate to obtain a mixture;
heating the mixture to a temperature in the range of 71-77° C.;
adding 17 wt. % of organic beeswax to the mixture and holding the temperature in the range of 71-77° C.;
adding 0.09 wt. % of thymol to the mixture and holding the temperature in the range of 71-77° C.;
adding 2.9 wt. % of menthol to the mixture and holding the temperature in the range of 71-77° C.;
adding 5.9 wt. % of camphor to the mixture and holding the temperature in the range of 71-77° C.;
after all the preceding steps have been performed:
adding 1.5 wt. % of vanilla oil to the mixture;
adding 0.025 wt. % or 25 mg of CBD microspheres to the mixture and holding the temperature in the range of 71-77° C.; and
letting the mixture set until reaching a semisolid state.
